(19) United States
(12) Patent Application Publication
(10) Pub. No.: US 2009/0155233 A1
(43) Pub. Date: Jun. 18, 2009

(54) METHOD FOR TREATING TUMOR USING IRRADIATED TUMOR CELL EXPRESSING HUMAN HEPATITIS B SURFACE ANTIGEN AND A PHARMACEUTICAL COMPOSITION COMPRISING THE TUMOR CELL

(76) Inventor: Seok Koo Cho, Seoul (KR)

Correspondence Address: Nixon Peabody LLP, 200 Page Mill Road, Suite 200, Palo Alto, CA, 94306, US

(21) Appl. No.: 12/293,819

(22) PCT Filed: Mar. 9, 2007
(86) PCT No.: PCT/KR2007/001157

§ 371 Date: Sep. 22, 2008

(30) Foreign Application Priority Data
Mar. 24, 2006 (KR) 10-2006-0026993
May 8, 2006 (KR) 10-2006-0041074

(51) Int. Cl. A61K 48/00

(52) U.S. Cl. 424/93.21

(57) ABSTRACT

The present invention relates to a method for treating a tumor in mammals comprising administering tumor cells in a therapeutically effective amount, in which the tumor cells lose their proliferative function by irradiation, are genetically manipulated to express hepatitis B surface antigen and are the same as the tumor to be treated, and a pharmaceutical composition for treatment of a tumor comprising the tumor cells.

### TECHNICAL FIELD

The present invention relates to a method for treating tumor using irradiated tumor cell expressing human hepatitis B surface antigen and a pharmaceutical composition for treating tumor comprising the tumor cell.

### BACKGROUND ART

Though it is presumed that a tumor specific antigen exists, effective and spontaneous immunization against tumor cells are not observed. This is because there is a immune-evasion mechanism associated with the tumor cells. As the immune-evasion mechanism, for example, downward control of MHC 1 expression in the tumor cells, immunity-deficient mutant, production of immune-suppressing cytokine are proposed (Gabrilovich D, Pisarev V. Curr Drug Targets 2003;4:525; Piemonti L, Zerbi A, Di Carlo V. Drugs Today (Barc) 2003;39:701). In order to stimulate anti-tumor immunity, irradiated tumor vaccine, mixed inoculation of tumor cells and pathogens, dendritic cells(DC)-based vaccination and therapy combined with cytokine are clinically applied (Baral R. Indian J Exp Biol 2005;43:389; Morisaki T, Matsumoto K, Onishi H, et al. Hum Cell 2003;16:175; Yannelli J R, Wroblewski J M. Vaccine 2004;23:97).

The renal carcinoma cell (RENCA) is resistant to chemo therapy and radiation therapy and often detected in the late state. At present, one method selected for treatment of renal cancer is kidney removal to cut off a large part of the kidney. The immunotherapy is one of promising methods but the early clinical test results are not satisfactory (Dillman R, Barth N, VanderMolen L, et al. Cancer Biother Radiopharm 2004;19:570; Tani K, Azuma M, Nakazaki Y, et al. Mol Ther 2004;10:799; Volk J, Sel S, Ganser A, Schoffski P. Curr Drug Targets 2002;3:401). Among the various strategies used to produce anti-tumor immunity against renal carcinoma cells, self-tumor vaccine is known as a low toxic, treatment. The most often side effects include topical erythema, topical pain arid fever. However, since the tumor growth can proceed faster as preparation takes a more time, the tumor cell vaccine has a defects in that it cannot use a sufficient amount of tumor cells (Dranoff G, Jaffee E, Lazenby A, et al. Proc Natl Acad Sci USA 1993;90:3539).

With contrast to the tumor cells, most of the viruses are strong inducers of cell-mediated immunity (Qiu S J, Lu L, Qiao C, et al. J Cancer Res Clin Oncol 2005;131:429; Restifo N P, Surman D R, Zheng H, Palese P, Rosenberg S A, Garcia-Sastre A. Virology 1998;249:89). Immunity against human hepatitis B surface antigen (HBsAg) has been used in the treatment of liver cancer after chronic HBV infection. The effect of HBsAg vaccination can be maintained in highly immune-suppressed cancer patients (Chisari F V. Rous-Whipple Am J Pathol 2000;156:1117). Moreover, HBsAg-specific reaction is connected with the in vitro T cell reaction to melanoma peptide and enhances general immune functions (Smithers M, O'Connell K, MacFadyen S, et al. Cancer Immunol Immunother 2003;52:41).

U.S. Pat. Nos. 5,637,483 and 5,904,920 disclosed methods for treating a tumor in mammals comprising administering tumor cells to the mammals, in which the tumor cells are obtained by irradiating radioactive rays such as gamma rays to tumor cells expressing GM-CSF to remove proliferative function. However, there has not been disclosed a method for treating a tumor in mammals using an HBsAg-expressing tumor cell.

### DISCLOSURE OF INVENTION

### Technical Problem

It is an object of the present invention to provide a method for treating tumor in mammals using irradiated tumor cells expressing human hepatitis B surface antigen (HBsAg).

It is another object of the present invention to provide a pharmaceutical composition for treatment of tumor comprising irradiated tumor cells expressing HBsAg.

### Technical Solution

Therefore, according to the present invention, there is provided a method for treating tumor in mammals comprising administering tumor cells in a therapeutically effective amount,

in which the tumor cells lose their proliferative function by irradiation, are genetically manipulated to express hepatitis B surface antigen and are the same as the tumor to be treated.

According to the present invention, the tumor cells may be transformed into a vector including nucleic acid coding hepatitis B surface antigen. The term “hepatitis B surface antigen (HBsAg)” used herein means a surface antigen of hepatitis B that induces immune response to hepatitis B in the human body. Hepatitis B surface antigen (HBsAg) is well known to the art and may have the sequence of NCBI GenBank accession No. X01587 (Fujiyama, A. et. al., J. Nucleic Acids Res. 11 (13), 4601-4610(1983)). The radioactive rays which are usable in the present invention include those which can remove proliferative function by inactivation of cells, for example ultraviolet rays and gamma rays, with preference being gamma rays. The time and conditions for the treatment of the radioactive rays can readily set up by those skilled in the art.

According to the present invention, the vector may include, for example, retroviral vector. The retroviral vector may be pMX-HBsAg-IRES-puro (deposited with Korean Culture Center of Microorganisms on Mar. 31, 2006 as Accession No. KCCM-10744P and deposited with Korean Cell Line Research Foundation on Mar. 30, 2006 as Accession No. KCLRF-BP-00131) having the vector map of FIG. 3, in which a polynucleotide having a nucleotide sequence of NCBI GenBank accession No. X01587is inserted into Xho1 and BamH1 sites of the pMX-IRES-puro vector but is not limited thereto. The retroviral vector may further comprise 5′ LTR and 3′ LTR, lack a complete gag, env, or pol gene and contain a functional selectable marker. The retrovirus may be packaged within the plat:E cell. In addition, vectors which can be used in the present invention include anyone of those known to the art to express HBsAg on the cell surface.

According to the present invention, the tumor may include melanoma or carcinoma but is not limited thereto. The carcinoma may be selected from the group consisting of renal cancer, lung cancer, rectal cancer, breast cancer and prostate cancer, with preference being renal cancer.

According to the present invention, the tumor cell may be administered in combination with other tumor cells in the same form as the tumor cell which lose their proliferative function by irradiation of radioactive rays but are not genetically manipulated to express hepatitis B surface antigen.

The ratio of the tumor cells expressing hepatitis B surface antigen to the tumor cells not expressing hepatitis B surface antigen may be preferably 1:1-5, but the present invention is not limited thereto. The ratio can be selected considering types of the tumor to be treated and the conditions of the patient. By administering a combination of two types of tumor cells, it is possible to increase the tumor treatment effect can be increased.

According to the present invention, the administration of the tumor cells can be performed by any method known to the art. For example, it includes intravascular, subcutaneous or intramuscular injection. The term therapeutically effective amount, used herein, means an amount inducing inhibition, regression, partial or complete removal of the tumor in the mammal and may be readily adjusted by a person skilled in the art. The term intends any reduction in the size, capacity, growing rate or shape of the existing tumor.

It is believed that the method according to the present invention is accomplished by administering the tumor cells according to the present invention to a mammal to induce systemic immune response in the mammal but the present invention is not limited to any specific mechanism.

In the method according to the present invention, the mammals may be any mammals, such as primates including for example, human and rodents. Preferably, the mammals include human, mouse, pig and cow but are not limited thereto. The mammals are preferably immunized with hepatitis B surface antigen and thus retain antibodies against hepatitis B surface antigen in the blood.

Also, according to the present invention, there is provided a pharmaceutical composition for treatment of a tumor in mammals comprising tumor cells which express hepatitis B surface antigen and lose their proliferative function by irradiation, and a pharmaceutically acceptable carrier.

In the pharmaceutical composition according to the present invention, the tumor cell may be transformed into a vector including nucleic acid coding hepatitis B surface antigen. The vector may be any one capable of expressing hepatitis B surface antigen in the tumor cell, including, for example, retroviral vector. Preferably, the retroviral vector may be pMX-HBsAg-IRES-puro (Accession No. KCCM-10744P, deposited on Mar. 31, 2006, and Accession No. KCLRF-BP-00131, deposited on Mar. 30, 2006) having the vector map of FIG. 3, in which a polynucleotide having a nucleotide sequence of NCBI GenBank accession No. X01587 is inserted into Xho1 and BamH1 sites of the pMX-IRES-puro vector but is not limited thereto.

The tumor to which the pharmaceutical composition can be applied may be melanoma or carcinoma. The carcinoma is selected from the group consisting of renal cancer, lung cancer, rectal cancer, breast cancer and prostate cancer, with preference being renal cancer. The terms and conditions which are not separately described for the pharmaceutical composition have the same meaning as those described for the method according to the present invention.

The composition according to the present invention may be administered through a route well-known to the art. For example, it can be directly administered to the subject by any route such as intravascular, intramuscular, oral, transdermal, mucosal intranasal, intratracheal or subcutaneous administration. The composition can be administered systemically or topically.

The composition according to the present invention can be formulated into an oral formulation such as granules, powder, solution, tablet, capsule or dry syrup, or a parentaral formulation such as injection without any limitation. Preferably, the composition according to the present invention is prepared in the form of solution or injection.

The composition according to the present invention may be administered in an amount of 1×10⁶ cells/kg to 5×10⁶ cells/kg without any limitation. The dosage may be properly adjusted considering types of the mammals to be treated and the state of the tumor.

According to the present invention, the mammals may be any mammals, such as primates including, for example, human and rodents. Preferably, the mammals include human, mouse, pig and cow but are not limited thereto. The mammals are preferably immunized with hepatitis B surface antigen and thus retain antibodies against hepatitis B surface antigen in the blood.

The pharmaceutically acceptable carrier used in the composition according to the present invention may be any one selected from a dilluent, an excipient, a disintegrant, a binder and a lubricant, or a mixture of two or more thereof. The composition may be in the form of solution and injection such as sterile aqueous solution, and contain 10 to 40% of propylene glycol and sodium chloride in an amount sufficient to prevent hemolysis (ex.: about 1%), as needed.

### Advantageous Effects

According to the present invention, it is possible to treat a tumor in mammals by using irradiated cancer cells expressing HBsAg, particularly, renal carcinoma cells.

The composition according to the present invention can effectively treat a tumor in mammals.

### BRIEF DESCRIPTION OF THE DRAWINGS

Further objects and advantages of the invention can be more fully understood from the following detailed description taken in conjunction with the accompanying drawings, in which:

FIG. 1 and FIG. 2 each show human hepatitis B surface antigen and its gene expressed from the renal carcinoma cell line transformed with pMX-HBsAg-IRES-puro; and

FIG. 3 shows the vector map of pMX-HBsAg-TRES-puro.

### BEST MODE FOR CARRYING OUT THE INVENTION

### 1. Materials and Method

(1) Mouse and Cell Line

BALB/c mouse (female, 6 weeks to 8 weeks old) was purchased from Samtago (Kwangju, Korea). The animal was kept in an animal farm set to a humidity of 55±5%, light (12/12 h light/dark), and a temperature of 22±1° C., under specific pathogen-free conditions. The air in the farm was filtered by a HEPA filter system to exclude bacteria and viruses. The mouse was freely accessed to food and water. All the experiment processes was performed following the guideline of NIH and Helsinki declaration and the use and management of the animal was approved by Korean Catholic University committee. RENCA renal adenocarcinoma cell line (renal adenocarcinoma) (the same species as BALB/c) was maintained in DMEM supplemented with 10% FCS and penicillin/streptomycin. For virus packaging, ecotropic packaging cell line Plat-E was donated by Dr. Toshio Kitamura in Tokyo University and maintained in DMEM supplemented with 10% FCS, 10/ml puromycin, and penicillin/streptomycin.

### EXAMPLE 1

### Preparation of Plasmid and Plasmid Transfaction

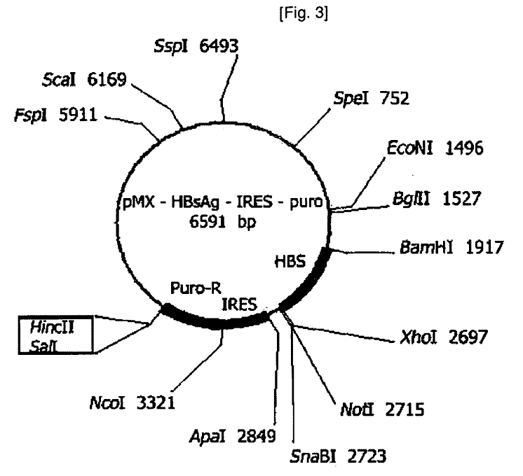

HBsAg gene with 681-bp length was amplified from full length hepatitis B virus genome of plasmid pBRHBadr72 (Japan Health Sciences Foundation, Tokyo, Japan) (Nucleic Acid Research, 11, 4601-4610, 1983) by PCR using 5′-CACCATGGAGAACACAACATCAGGATT-3′ (forward: SEQ NO: 1) and 5′-TTAAATGTATACCCAAAGACAAA-3′ (reverse: SEQ. NO: 2) as primers and inserted into Xho1 and BamH1 sites of retroviral vector pMX-IRES-puro (provided by Dr. Toshio Kitamura in Tokyo University. FIG. 3 shows the vector map of pMX-HBsAg-IRES-puro. The recombinant plasmid (pMX-HBsAg-IRES-puro) or a blank plasmid (pMX-IRES-puro) was transfacted into Plat-E cell using Fugene 6 (Boehringer Mannheim, German). As for the plat-E cell, see Morita S, Kojima T, Kitamura T. Plat-E: an efficient and stable system for transient packaging of retroviruses. Gene Ther 2000;7:1063, full text of which is incorporated herein by reference.

### EXAMPLE 2

### Retrovirus Gene Delivery and Stable Cell Line

After 48 hours from the transfaction to Plat-E, the collected supernatant was added to the RENCA cell culture to deliver the gene. The cell line stably expressing HBsAg was chosen and grown in a culture medium containing 10/ml puromycin.

### EXAMPLE 3

### Vaccination and Tumor Induction Stratage

The animal was actively immunized with recombinant HBsAg (10 i.p. injection, Hepavax; Green Cross Vaccine, Yongin, Korea) or vehicle and boosted every three days until the tumor inoculation. After one week from the inoculation of the protein vaccine, the stably expressing RENCA cell line was irradiated by a source of 50-Gy¹³⁷ Cs radioactive rays to prepare a tumor vaccine without proliferative function. The tumor vaccine was intradermally injected to the mouse in an amount of 5 to 10×10⁴tumor cells/mouse.

After two weeks from the inoculation of the protein, the tumor cells were subcutaneously inoculated to the mouse in an amount of 1 to 5×10⁶ cells/mouse. The anti-HBsAg antibodies produced in the mouse was detected using Enzygnost™ HBsAg 5.0 (Dade Boehringer, Marburg, Germany) with a cutoff value of 0.115 O.D. and a maximum value of 4.0 O.D.

### Result

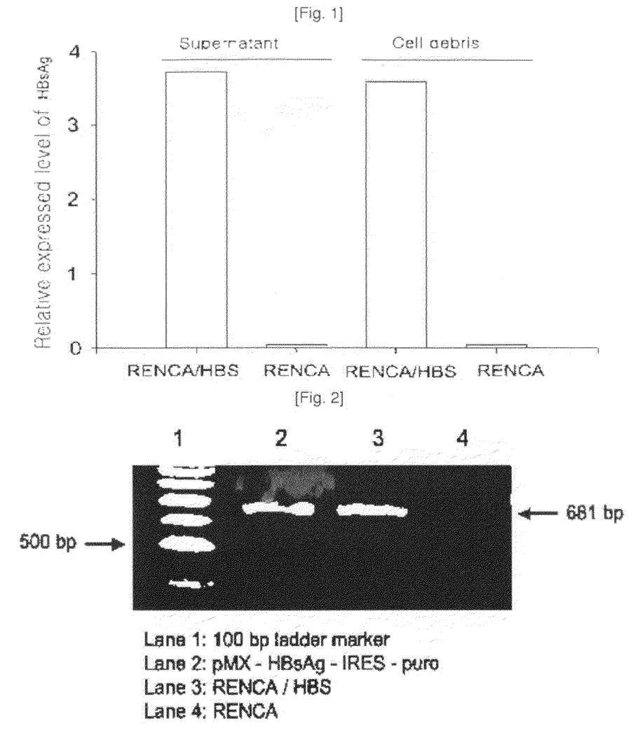

The RENCA cell line was transformed with pMX-HBsAg-IRES-puro to produce the HBsAg-expressing RENCA cell line. The HBsAg protein and genome DNA obtained from the HBsAg-expressing RENCA cell line was analyzed by ELISA and PCR (FIGS. 1 and 2). The recombinant HBsAg was stably expressed in the transformed RENCA cell and the recombinant HBsAg was used in all of the subsequent experiments. RENCA transformed with pMX-HBsAg-IRES-puro is referred to as RENCA/HBS, hereinafter. FIGS. 1 and 2 each show human hepatitis B surface antigen (HBsAg) and its gene expressed from the renal carcinoma cell line transformed with pMX-HBsAg-IRES-puro. As shown in FIG. 2, pMX-HBsAg-IRES-puro and DNA product amplified from HBsAg gene of the renal carcinoma cell line transformed with pMX-HBsAg-IRES-puro were 681 bp.

Before examining the inoculation of tumor vaccine, the present inventors examined anti-tumor activity of the HBsAg immunization. The RENCA/HBS cell was completely susceptible to the HBsAg immunization while all the other groups produced a tumor (See Table 1).

TABLE 1
Effect of rHBsAg vaccination on tumor formation after RENCA or
RENCA/HBS challenge

| rHBsAg | Tumor vaccination | Tumor challenge | Tumor occurrence |
|---|---|---|---|
| vaccination | (PBS inoculation) | (10⁶) | in mouse |
| − | PBS | RENCA | 10/10 |
| − | PBS | RENCA/HBS | 10/10 |
| + | PBS | RENCA | 10/10 |
| + | PBS | RENCA/HBS | 0/10 |

This means that the inoculation of HBsAg vaccine induces specific anti-tumor immunization against HBsAg-expressing cancer cells. However, in respect of the kidney cell carcinoma, the anti-tumor immunization is not accomplished only by boosting the HBsAg specific immune response.

In order to prepare the tumor vaccine, the RENCA/HBS and RENCA cells were irradiated by radioactive rays so as to avoid risks of malignant expression type derived from the living cancer vaccine. In order to copy human HBsAg immunization, after HBsAg vaccination, the mouse was administered with irradiated RENCA (10⁵ cell) or irradiated RENCA mixture (5×10⁴RENCA/HBS and 5×10⁴ RENCA), and then injected with 10⁶ RENCA cells to induce tumor. Unexpectedly, both the RENCA and RENCA mixture prevented RENCA-induced tumor formation in the mouse (Table 2).

TABLE 2
Effect of tumor vaccine connected with HBsAg immunization on
tumor formation by RENCA

| rHBsAg | | Tumor challenge | Tumor occurrence |
|---|---|---|---|
| vaccination | Tumor vaccination | (10⁶) | in mouse |
| + | RENCA | RENCA | 0/5 |
| | (10⁵) | | |
| + | RENCA/HBS | RENCA | 0/5 |
| | (5 × 10⁴) + | | |
| | RENCA (5 × 10⁴) | | |

Dranoff et al. have reported a similar effect in irradiated GM-CSF-transformed B16melanoma cell (Dranoff G, Jaffee E, Lazenby A, et al. Proc Natl Acad Sci USA 1993;90:3539). However, the tumor challenge of Dranoff et al. was the same as the amount used in the tumor vaccination and it was impossible to distinguish the relative anti-tumor activity of GM-CSF-transformed RENCA vaccination from the non-specific activity of the RENCA vaccine in an excessive amount (Kerkmann-Tucek A, Banat G A, Cochlovius B, Zoller M. Int J Cancer 1998;77:114). Since further less tumor load could be overcome only by RENCA vaccination, in a further more tumor load (5×10⁶ cells), as shown in Table 2, the present inventors could identify the effect of the RENCA/HBS vaccination from that of RENCA. The present inventors used an optimized ratio by increasing the challenge to 5×10⁶ RENCA cells and reducing the tumor vaccine to 5×10⁴ cells in the subsequent experiments.

Also, the effect of the RENCA/HBS vaccination on the RENCA/HBS tumor load was examined. Along with the HBsAg vaccination, the RENCA/HBS delayed the tumor formation after the RENCA/HBS challenge (See Table 3).

TABLE 3
Effect of tumor vaccination connected with HBaAg immunization
at optimized amount on tumor formation by RENCA or RENCA/HBS

| | | | Tumor |
|---|---|---|---|
| rHBaAg | Tumor vaccination | Tumor challenge | occurrence in |
| vaccination | (5 × 10⁴) | (5 × 10⁶) | mouse |
| + | PBS | RENCA/HBS | 2/6 |
| − | RENCA/HBS | RENCA/HBS | 4/6 |
| + | RENCA/HBS | RENCA/HBS | 0/6 |
| − | PBS | RENCA | 10/10 |
| + | RENCA | RENCA | 10/10 |
| + | ½ RENCA/HBS + | RENCA | 4/10 |
| | ½ RENCA | | |

After the HBsAg vaccination, the inoculation of the RENCA/HBS tumor vaccine successively suppressed the same tumor load (RENCA/HBS). Then, the present inventors examined if the RENCA/HBS tumor vaccination induced anti-tumor activity against only RENCA. The present inventors inoculated the mouse with the RENCA/HBS tumor vaccine in an optimized amount and injected RENCA tumor cells not-expressing HBsAg. As a result, the RENCA/HBS tumor vaccine significantly decreased the tumor mediated by RENCA but the RENCA tumor vaccine did not affect on the tumor (See Table 3).

### Discussion

The renal carcinoma cells are known to remove tumor-associated antigen due to their weak immunity. According to the clinical test and studies using MHC, B7.1 or cytokine-transfacted renal carcinoma cells, the assumption of weak immunity was strengthened (Marti W R, Oertli D, Meko J B, Norton J A, Tsung K. J Immunol Methods 1997;200:191; Hodge J W, Abrams S, Schlom J, Kantor J A. Cancer Res 1994;54:5552; Simons J W, Jaffee E M, Weber C E, et al. Cancer Res 1997;57:1537).

On the contrary, high-dose renal carcinoma cell vaccination prevented tumor formation, which means that the renal carcinoma cells have potential immunity. However, clinically, since the tumor growth is too fast, it is difficult to prepare a patient-tailored self-tumor vaccine. Therefore, it is desired to have a more efficient rumor vaccination strategy such as a much lower dose antigen-pulsed dendritic cells and cytokine gene-transformed tumor cells.

It is believed that the method according to the present invention is operated by the following mechanism but the present invention is not limited such a specific theory. Firstly, the particulate HBsAg antigen can promotes a specific immune response against several presumed renal carcinoma cell associated antigens. The recombinant HBsAg vaccination can improved immuno-recognition of the RENCA/HBS tumor vaccine, whereby anti-tumor immunity against several presumed tumor antigens can be induced when a subjected is challenged by RENCA.

Because of the bystander activation of the anti-tumor immunization by HBsAg, the method according to the present invention is very useful strategy to treat renal carcinoma cells when a patient maintains continuous immunity against HBsAg.

Secondarily, HBsAg enhances general immune function, thereby contributing the anti-tumor immunization against the renal carcinoma cells. The immune-suppression induced by the tumor begins topically in the renal carcinoma cells (Riccobon A, Gunelli R, Ridolfi R, et al. Cancer Invest 2004;22:871). Signal activating molecules such as T cell receptor zeta and epsilon chain and p561ck tyrosine kinase are expressed at a low level in tumor-infiltrating lymphocytes as compared to lymphocyte near tumor tissue or peripheral blood of the renal cancer patient.

Further, the renal cancer patient shows various immuno-deficiency, thereby developing serious complications as the disease proceeds (Elsasser-Beile U, Gierschner D, Welchner T, Wetterauer U. Anticancer Res 2003;23:433; Gratama J W, Zea A H, Bolhuis R L, Ochoa A C. Cancer Immunol Immunother 1999;48:263-1; Shabtai M, Ye H, Kono K, et al. Urol Oncol 2003;21:27). This can be connected to the method according to the present invention which can inhibit the development of renal cancer by enhancing the general immunity by HBsAg. It is believed that HBsAg enhances general immune functions, whereby the irradiated renal carcinoma cells expressing HBsAg allow the enhanced immune system to recognize adjacent renal cancer antigen as well as HBsAg.

### INDUSTRIAL APPLICABILITY

In sum, the vaccination of HBsAg and irradiated renal carcinoma cells expressing HBsAg to a subject promotes specific immune response to a presumed tumor vaccine or enhances general immunity, thereby improving the general anti-tumor immunity in an immune-suppressed renal cancer patient.

1. A method for treating tumor in mammals comprising administering tumor cells in a therapeutically effective amount, in which the tumor cells lose their proliferative function by irradiation, are genetically manipulated to express hepatitis B surface antigen and are the same as the tumor to be treated.

2. The method according to claim 1, in which the tumor cells are transformed by a vector comprising nucleic acid coding hepatitis B surface antigen.

3. The method according to claim 2, in which the vector is retroviral vector.

4. The method according to claim 3, in which the retroviral vector is pMX-HBsAg-IRES-puro (accession No. KCCM-10744P) having the vector map of FIG. 3, in which a polynucleotide having a nucleotide sequence of NCBI Genbank accession No. X01587 is inserted into Xho1 and BamH1 sites of pMX-IRES-puro vector.

5. The method according to claim 1, in which the tumor is melanoma or carcinoma.

6. The method according to claim 5, in which the carcinoma is selected from the group consisting of renal cancelling cancer, rectal cancer, breast cancer and prostate cancer.

7. The method according to claim 1, in which the tumor cells are administered in combination with other tumor cells in the same form as the tumor cell which lose their proliferative function by irradiation but are not genetically manipulated to express hepatitis B surface antigen.

8. The method according to claim 7, in which the ratio of the tumor cells expressing hepatitis B surface antigen to the tumor cells not expressing hepatitis B surface antigen is 1:1-5.

9. A pharmaceutical composition for treatment of a tumor in mammals comprising tumor cells which express hepatitis B surface antigen and lose their proliferative function by irradiation, and a pharmaceutically acceptable carrier.

10. The composition according to claim 9, in which the tumor cells are transformed by a vector comprising nucleic acid coding hepatitis B surface antigen.

11. The composition according to claim 10, in which the vector is retroviral vector.

12. The composition according to claim 11, in which the retroviral vector is pMX-HBsAg-IRES-puro (accession No. KCCM-10744P) having the vector map of FIG. 3, in which a polynucleotide having a nucleotide sequence of NCBI GenBank accession No. X01587 is inserted into Xho1 and BamH1 sites of pMX-IRES-puro vector.

13. The composition according to claim 9, in which the tumor is melanoma or carcinoma.

14. The composition according to claim 13, in which the carcinoma is selected from the group consisting of renal cancer, lung cancer, rectal cancer, breast cancer and prostate cancer.

15. The method according to claim 1, in which the mammals are inoculated with hepatitis B surface antigen to produce antibody against hepatitis B surface antigen.

16. The composition according to claim 9, in which the mammals are inoculated with hepatitis B surface antigen to produce hepatitis B surface antigen.


SEQUENCE LISTING (2 sequences)

SEQ ID NO: 1 - DNA, 27 bp, Artificial Sequence
  forward primer for HBsAg gene amplification
caccatggagaacacaacatcaggatt

SEQ ID NO: 2 - DNA, 23 bp, Artificial Sequence
  reverse primer for HBsAg gene amplification
ttaaatgtatacccaaagacaaa